(19) United States
(12) Patent Application Publication
(10) Pub. No.: US 2026/0021470 A1
(43) Pub. Date: Jan. 22, 2026

(54) Composition of Super Absorbent Polymer and Preparation Method Thereof

(71) Applicant: LG Chem, Ltd., Seoul (KR)

(72) Inventors: Ji Hong Min, Daejeon (KR); Jun Kyu Kim, Daejeon (KR)

(73) Assignee: LG Chem, Ltd., Seoul (KR)

(21) Appl. No.: 18/842,571

(22) PCT Filed: Mar. 24, 2023
(86) PCT No.: PCT/KR2023/003922

§ 371 Date: Aug. 29, 2024

(30) Foreign Application Priority Data
Mar. 29, 2022 (KR) 10-2022-0039062
Mar. 23, 2023 (KR) 10-2023-0038011

(51) Int. Cl. B01J 20/26; A61L 15/20; A61L 15/24; A61L 15/46; A61L 15/60; B01J 20/30

(52) U.S. Cl. CPC B01J 20/267; A61L 15/20; A61L 15/24; A61L 15/46; A61L 15/60; B01J 20/3014; B01J 20/3078; B01J 2220/44; B01J 2220/68

(57) ABSTRACT

The present disclosure relates to a composition of super absorbent polymer and a preparation method thereof, and more specifically, a composition of super absorbent polymer and a preparation method thereof, that may improve deodorization capacity and odor masking capacity by using specific additives in combination, to effectively inhibit odor generated from urine, and the like, when applied to a product such as a diaper, and the like, and bacteria growth during wearing of a product.

### CROSS-REFERENCE TO RELATED APPLICATIONS

The present application is a national phase entry under 35 U.S.C. § 371 of International Application No. PCT/KR2023/003922 filed Mar. 24, 2023, which claims priority from Korean Patent Application No. 10-2022-0039062 filed on Mar. 29, 2022, and Korean Patent Application No. 10-2023-0038011 filed on Mar. 23, 2023, with the Korean Intellectual Property Office, the disclosures of which are herein incorporated by reference in their entirety.

### TECHNICAL FIELD

The present disclosure relates to a composition of super absorbent polymer and a preparation method thereof. More specifically, the present disclosure relates to a composition of super absorbent polymer and a preparation method thereof, that may improve deodorization capacity and odor masking capacity by using specific additives in combination, to effectively inhibit odor generated from urine, and the like, when applied to a product such as a diaper, and the like, and bacteria growth during wearing of a product.

### BACKGROUND

A super absorbent polymer (SAP) is a synthetic polymeric material that can absorb moisture in an amount of about 500 to 1000 times its own weight. Various manufacturers have denominated it different names, such as a super absorbency material (SAM) and an absorbent gel material (AGM). Although such superabsorbent polymer started to be commercialized as sanitary items, now they have been used as water-holding materials for soil, water stop materials for civil engineering and architecture, sheets for raising seedings, freshness preservatives in the field of food circulation, fomentation material, etc., besides hygienic goods such as a disposable diaper for children, and the like.

In most cases, such superabsorbent polymer is being widely used in the field of hygienic goods such as diapers or sanitary pad, and the like. In the hygienic goods, the superabsorbent polymer is generally spread in pulp and included. However, recently, to provide hygienic goods such as diapers having thinner thickness, development of diapers having decreased pulp content, or even pulpless diapers in which pulp is not used at all, is being actively progressed.

As such, in the case of hygienic goods having decreased pulp, or pulpless hygienic goods, superabsorbent polymer is included at relatively high ratio, and such superabsorbent polymer particles are inevitably included in multilayers. The superabsorbent polymer should basically exhibit high absorption performance and absorption speed so that the entire superabsorbent polymer particles included in multilayers can more efficiently absorb liquid such as urine. Moreover, absorbed liquid should not be leaked even under external pressure, and besides, the superabsorbent polymer should have permeability so as to properly maintain the original shape even when it absorbs liquid and is swollen. Thus, a lot of research is being conducted, such as for surface crosslinking, for improving basic absorption force and water holding capacity.

Meanwhile, super absorbent polymer may be used in hygienic products, and in this case, due to the odor of absorbed liquids such as excretion of human and pet, afterfeel may be degraded. Further, bacteria growth is accelerated as the wearing time passes, thus generating odor.

Thus, demand for odor inhibition as well as basic properties of super absorbent polymer including absorption power and water holding capacity is gradually increasing. Thus, preparation of super absorbent polymer that has deodorization capacity and can effectively inhibit bacteria growth, is needed.

### Technical Problem

It is an object of the present disclosure to provide a composition of super absorbent polymer and a preparation method thereof. More specifically, it is an object of the present disclosure to provide a composition of super absorbent polymer and a preparation method thereof, that improves deodorization capacity and odor masking capacity by using specific additives in combination, to effectively inhibit odor such as urine and bacteria growth when applied to a product such as a diaper, and the like.

### Technical Solution

To solve the objects, according to the present disclosure, there is provided a composition of super absorbent polymer comprising:
super absorbent polymer comprising a base resin comprising crosslinked polymer formed by crosslinking polymerization of acrylic acid-based monomers having at least partially neutralized acid groups and an internal crosslinking agent, and a surface crosslink layer formed on the surface of the base resin, in which the crosslinked polymer is additionally crosslinked by a surface crosslinking agent;
an amino acetate-based chelating agent; and
cysteine,
wherein the amino acetate-based chelating agent and cysteine are each independently included inside the surface crosslink layer or on the surface of the surface crosslink layer.

According to the present disclosure, there is also provided a method for preparing a composition of super absorbent polymer comprising steps of:
subjecting acrylic acid-based monomers having at least partially neutralized acid groups to crosslinking polymerization, in the presence of an internal crosslinking agent and a polymerization initiator, to form hydrogel polymer (step 1);
preparing base resin comprising crosslinked polymer obtained by drying and grinding the hydrogel polymer (step 2);
mixing a surface crosslinking composition with the base resin to prepare a mixture (step 3); and
heat treating the mixture to prepare super absorbent polymer in which a surface crosslink layer is formed on the surface of the base resin (step 4),
wherein an amino acetate-based chelating agent and cysteine are each independently mixed in at least one steps of the steps 2 to 4 and steps before and after them.

### Technical Effects

As explained above, the present disclosure is characterized by providing a composition of super absorbent polymer that has excellent deodorization capacity by combined use of specific additives in super absorbent polymer, and inhibits bacteria growth to inhibit generation of additional odor, and a preparation method thereof.

### DETAILED DESCRIPTION

The terms used herein are only to explain specific embodiments and are not intended to limit the present disclosure.

A singular expression includes a plural expression thereof, unless the context clearly indicates otherwise. Throughout the specification, the terms “comprise”, “equipped” or “have”, etc. are intended to designate the existence of practiced characteristic, number, step, constructional element or combinations thereof, and they are not intended to preclude the possibility of existence or addition of one or more other characteristics, numbers, steps, constructional elements or combinations thereof.

The terms ‘first’, ‘second’, ‘third’ and the like are used to explain various constructional elements, and they are used only to distinguish one constructional element from the other constructional elements.

As used herein, the term “polymer” means a polymerized state of water soluble ethylenically unsaturated monomers, and may include polymers of all moisture content ranges or all particle diameter ranges. Among the polymers, polymer that is not dried and has a moisture content of about 40 wt % or more, may be referred to as hydrogel polymer, and ground and dried particles of such hydrogel polymer may be referred to as crosslinked polymer.

Further, as used herein, “base resin” or “base resin powder” means particle or powder obtained by drying and grinding polymer polymerized from acrylic acid-based monomers, before passing through surface modification or surface crosslinking step as described later.

Further, the term “super absorbent polymer” or “super absorbent polymer powder” means crosslinked polymer polymerized from water soluble ethylenically unsaturated monomers including acid groups, at least a part of said acid groups being neutralized, or base resin powder consisting of super absorbent polymer particles obtained by grinding the crosslinked polymer, or it is used to include the crosslinked polymer or base resin made suitable for productization by subjecting it to additional processes, for example, surface crosslinking, fine powder reassembling, drying, grinding, classification, and the like.

Although various modifications can be made to the present disclosure and the present disclosure may have various forms, specific examples will be illustrated and explained in detail below. However, it should be understood that it is not intended to limit the present disclosure to specific disclosure, and that the present disclosure includes all the modifications, equivalents or replacements thereof without departing from the spirit and technical scope of the present disclosure.

Hereinafter, a method for preparing super absorbent polymer and super absorbent polymer according to specific embodiments of the present disclosure will be explained in detail.

### (Composition of Super Absorbent Polymer)

According to one embodiment of the disclosure, there is provided a composition of super absorbent polymer.

The super absorbent polymer comprises a base resin comprising a crosslinked polymer formed by crosslinking polymerization of acrylic acid-based monomers having at least partially neutralized acid groups and an internal crosslinking agent, and a surface crosslink layer formed on the surface of the base resin, in which the crosslinked polymer is additionally crosslinked by a surface crosslinking agent; an amino acetate-based chelating agent; and cysteine, wherein the amino acetate-based chelating agent and cysteine are each independently included inside the surface crosslink layer or on the surface of the surface crosslink layer.

Super absorbent polymers are variously used in hygienic products such as diapers, sanitary pads, and the like, and in this case, due to the odor of excretion of human and pet, afterfeel may be degraded. Further, as the wearing time passes, bacteria growth is accelerated by the liquid absorbed in the product, thus generating additional odor.

Deodorant substances previously included in a product to reduce odor should be excessively used to realize deodorization capacity of an aimed degree, and thus, absorption properties may be significantly deteriorated, and production cost may increase.

Thus, the inventors of the present disclosure found out that by using specific additives in combination, odor generated by various causes may be effectively controlled without deterioration of basic properties of super absorbent polymer including absorption force and water holding capacity.

Specifically, according to the present disclosure, both primary odor resulting from odor-generating substance included in body fluid, urine, and the like, when the product is applied, and secondary odor resulting from bacteria growth can be effectively controlled. Moreover, by combined use of two kinds of additives, excellent deodorization capacity and odor masking capacity can be realized with relatively small amount, which is economical, and particularly, does not reduce absorption properties.

The amino acetate-based chelating agent is a component capable of effectively inhibiting the growth of bacteria such as E. coli generated by odor-generating substance. As the wearing time of a product passes, bacteria growth is accelerated by the odor-generating substance remaining in the product, and thus, additional odor is generated, but the amino acetate-based chelating agent inhibits such bacteria growth to effectively reduce additional odor generation.

Particularly, the amino acetate-based chelating agent is a divalent cation forming a salt crosslink between the constituents of cell membrane, and it can effectively inhibit bacteria growth by destructing the cell membrane of bacteria.

The amino acetate-based chelating agent may include, for example, one or more selected from the group consisting of ethylenediamine tetraacetic acid (EDTA), L-glutamic acid diacetate (GLDA), methyl glycine diacetic acid (MGDA), hydroxyethyl ethylenediamine triacetic acid (HEDTA), ethanol diglycinic acid (EDG), diethylenetriamine pentaacetic acid (DTPA), and salts thereof.

The amino acetate-based chelating agent may be included in the content of 0.01 to 3 parts by weight, based on 100 parts by weight of the base resin, and preferably, may be included in the content of 0.025 parts by weight or more, 0.05 parts by weight or more, 0.1 parts by weight or more, 2.5 parts by weight or less, 2.0 parts by weight or less, 1.5 parts by weight or less, or 0.01 to 2.5 parts by weight, 0.01 to 2.0 parts by weight, 0.025 to 3.0 parts by weight, 0.025 to 2.5 parts by weight, 0.05 to 2.5 parts by weight, 0.05 to 2.0 parts by weight, 0.1 to 2.5 parts by weight, or 0.1 to 2.0 parts by weight.

If used in the above content range, it can effectively inhibit bacteria without deterioration of absorption properties, and thus, remarkably improve deodorization capacity and odor masking capacity of absorbent polymer. The chelating agent may be mixed and used in the form of a salt with an aqueous solution, and thus, the content range is based on the solid content. Meanwhile, in case the chelating agent is included in a small amount, bacteria inhibition property of an aimed degree may not be realized, and in case included in an excessive amount, unique properties of super absorbent polymer may be deteriorated.

The cysteine may chemically react with odor-generating substance to reduce odor. The odor-generating substance generally has small molecular weight, and it reacts with cysteine, and thereby, odor may be reduced or removed, and thus, effectively inhibited. Particularly, cysteine is effective for reducing odor generated from aldehydes and ketones. Meanwhile, in case similar amino acid methionine, and the like, are used, deodorization capacity may be exhibited to some degree, but they have different main functional groups, and thus, even if applied to SAP in the same amount, absorption properties and deodorization capacity may be remarkably deteriorated, compared to cysteine.

The cysteine may be included in the content of 0.01 to 3 parts by weight, based on 100 parts by weight of the based resin, and preferably, it may be included in the content of 0.025 parts by weight or more, 0.05 parts by weight or more, 0.1 parts by weight or more, 2.5 parts by weight or less, 2.0 parts by weight or less, 1.5 parts by weight or less, or 0.01 to 2.5 parts by weight, 0.01 to 2.0 parts by weight, 0.025 to 3.0 parts by weight, 0.025 to 2.5 parts by weight, 0.05 to 2.5 parts by weight, 0.05 to 2.0 parts by weight, 0.1 to 2.5 parts by weight, or 0.1 to 2.0 parts by weight.

When the cysteine is used in the above content range, it may effectively inhibit odor generation without deteriorating absorption properties, thus remarkably improving deodorization capacity and odor masking capacity of absorbent polymer. The cysteine may be mixed and used in the form of a salt with an aqueous solution, and thus, the content range is based on the solid content. Meanwhile, if the cysteine is included in a small amount, deodorization property may not be realized, and if included in an excessive amount, unique properties of super absorbent polymer may be deteriorated, and there may be process problems due to incomplete dissolving.

The base resin comprises crosslinked polymer formed by crosslinking polymerization of acrylic acid-based monomers having at least partially neutralized acid groups and an internal crosslinking agent

The acrylic acid-based monomers may be any monomers commonly used in the preparation of super absorbent polymer. As non-limiting examples, the acrylic acid-based monomer may be a compound represented by the following Chemical Formula 1:

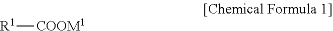

In the Chemical Formula 1,
R¹ is a C2-5 alkyl group comprising an unsaturated bond,
M¹ is a hydrogen atom, a monovalent or divalent metal, an ammonium group or an organic amine salt.

Preferably, the monomers may be one or more selected from the group consisting of acrylic acid, methacrylic acid, and monovalent metal salts, divalent metal salts, ammonium salts and organic amine salts thereof.

The acrylic acid-based monomers may have acid groups, and at least a part of the acid groups may be neutralized. Preferably, the monomers partially neutralized with alkali substance such as sodium hydroxide, potassium hydroxide, ammonium hydroxide and the like, may be used.

Wherein, the degree of neutralization of the monomers may be 40 to 95 mol %, or 40 to 80 mol %, or 45 to 75 mol %. Although the range of the neutralization degree may vary according to the final properties, if the neutralization degree is too high, neutralized monomers may be precipitated, and thus, it may be difficult to smoothly progress polymerization, and to the contrary, if the neutralization degree is too low, absorption force of polymer may be significantly lowered, and it may exhibit elastic rubber-like properties, which is difficult to handle.

The “internal crosslinking agent” is used to distinguish it from a “surface crosslinking agent” for crosslinking the surface of base resin, and it functions for crosslinking the unsaturated bonds of the above-explained acryl-based monomers to polymerize. Crosslinking in this step progresses without distinction of the surface or inside, but by the surface crosslinking process of base resin as described later, the surface of the finally prepared super absorbent polymer particle consists of a structure crosslinked by the surface crosslinking agent, and the inside consists of a structure crosslinked by the internal crosslinking agent.

As the internal crosslinking agent, any compounds can be used so long as they enable the introduction of crosslink during polymerization of the acrylic acid-based monomers. As non-limiting examples, as the internal crosslinking agent, multifunctional crosslinking agents such as N,N′-methylenebisacrylamide, trimethylolpropane tri(meth)acrylate, ethyleneglycol di(meth)acrylate, polyethyleneglycol (meth)acrylate, propyleneglycol di(meth)acrylate, polypropyleneglycol (meth)acrylate, butanediol di(meth)acrylate, butyleneglycol di(meth)acrylate, diethyleneglycol di(meth)acrylate, hexanediol di(meth)acrylate, triethyleneglycol di(meth)acrylate, tripropyleneglycol di(meth)acrylate, tetraethyleneglycol di(meth)acrylate, dipentaerythritol pentaacrylate, glycerin tri(meth)acrylate, pentaerythritol tetraacrylate, triallylamine, ethyleneglycol diglycidyl ether, propyleneglycol, glycerin, or ethylenecarbonate may be used alone or in combinations, but it is not limited thereto.

Such an internal crosslinking agent may be used at the concentration of 0.001 to 1 wt %, or 0.01 to 0.8 wt %, or 0.1 to 0.7 wt %, based on the monomer composition. If the concentration of the internal crosslinking agent is too low, absorption speed of polymer may decrease, and gel strength may become weak. To the contrary, if the concentration of the internal crosslinking agent is too high, absorption force of superabsorbent polymer may decrease, and thus, it may not be preferable as an absorbent.

Besides, the monomer composition may further comprise additives such as a thickener, a plasticizer, a preservation stabilizer, an antioxidant, etc., as necessary.

The super absorbent polymer comprises a surface crosslink layer formed on the surface of the base resin, in which the crosslinked polymer is additionally crosslinked by a surface crosslinking agent.

Wherein, the amino acetate-based chelating agent and cysteine are each independently included inside the surface crosslink layer or on the surface of the surface crosslink layer. It means that the amino acetate-based chelating agent and cysteine are each independently added after polymerization of base resin (i.e., before a surface crosslinking step), during the surface crosslinking step, or after the surface crosslinking step, which will be explained in detail in the preparation method of a composition of super absorbent polymer as described later.

The surface crosslink layer is formed by additional crosslinking of the crosslinked polymer by a surface crosslinking agent, wherein the surface crosslinking agent is not specifically limited so long as it is commonly used for surface crosslinking of super absorbent polymer and is a compound capable of reacting with the functional groups of the polymer.

Preferably, to improve the properties of produced super absorbent polymer, as the surface crosslinking agent, one or more selected from the group consisting of polyhydric alcohol compounds; epoxy compounds; polyamine compounds; haloepoxy compounds; condensation products of haloepoxy compounds; oxazoline compounds; mono-, di- or polyoxazolidinone compounds; cyclic urea compounds; multivalent metal salts; and alkylene carbonate compounds may be used.

Specifically, as the examples of the polyhydric alcohol compound, one or more selected from the group consisting of mono-, di-, tri-tetra- or polyethylene glycol, monopropylene glycol, 1,3-propanediol, dipropylene glycol, 2,3,4-trimethyl-1,3-pentanediol, polypropylene glycol, glycerol, polyglycerol, 2-butene-1,4-diol, 1,4-butanediol, 1,3-butanediol, 1,5-pentanediol, 1,6-hexandiol, and 1,2-cyclohexanedimethanol may be used.

Further, as the epoxy compound, ethylene glycol diglycidyl ether and glycidol, and the like may be used, and as the polyamine compound, one or more selected from the group consisting of ethylenediamine, diethylenetriamine, triethylenetriamine, tetraethylenepentamine, pentaethylenehexamine, polyethyleneamine, and polyamidepolyamine may be used.

Further, as the haloepoxy compound, epichlorohydrin, epibromohydrin, and α-methylepichlorohydrin may be used, and as the mono-, di- or polyoxazolidinone compound, for example, 2-oxazolidinone, and the like may be used.

Further, as the alkylene carbonate compound, ethylene carbonate, and the like may be used. These compounds may be used alone or in combinations. Meanwhile, to increase the efficiency of the surface crosslinking process, among these surface crosslinking agents, one or more kinds of C2-10 polyhydric alcohol compounds may be included.

The content of the surface crosslinking agent added may be appropriately selected according to the kind of the surface crosslinking agent added and reaction conditions, but commonly, it may be used in the amount of about 0.001 to about 5 parts by weight, preferably about 0.01 to about 3 parts by weight, more preferably about 0.05 to about 2 parts by weight, based on 100 parts by weight of the polymer.

If the content of the surface crosslinking agent is too small, a surface crosslinking reaction may hardly occur, and if it is greater than 5 parts by weight, based on 100 parts by weight of the polymer, due to the excessive progression of surface crosslinking reaction, absorption capacity and properties may be deteriorated.

Meanwhile, the surface crosslinking agent may further comprise inorganic materials. As such inorganic material, one or more selected from the group consisting of silica, clay, alumina, silica-alumina composite material, titania, zinc oxide and aluminum sulfate may be used. The inorganic material may be used in the form of powder or liquid, and particularly, alumina powder, silica-alumina powder, titania powder, or a nano silica solution. The inorganic material may be used in the content of about 0.001 to about 1 part by weight, based on 100 parts by weight of the base resin.

Further, the surface crosslinking agent may further comprise a thickener. If the surface of base resin powder is additionally crosslinked in the presence of a thickener, property deterioration may be minimized even after grinding. Specifically, as the thickener, one or more selected from polysaccharide and hydroxy-containing polymer may be used. As the polysaccharide, a gum-based thickener and a cellulose-based thickener, and the like may be used. As specific examples of the gum-based thickener, xanthan gum, arabic gum, karaya gum, tragacanth gum, ghatti gum, guar gum, locust bean gum and psyllium seed gum, and the like may be mentioned, and as specific examples of the cellulose-based thickener, hyrdoxypropylmethylcellulose, carboxymethylcellulose, methylcellulose, hydroxymethylcellulose, hydroxyethylcellulose, hydroxypropylcellulose, hydroxyethylmethylcellulose, hydroxymethylpropylcellulose, hydroxyethylhydroxypropylcellulose, ethylhydroxyethylcellulose and methylhydroxypropylcellulose, and the like may be mentioned. Meanwhile, as specific examples of the hydroxy-containing polymer, polyethyleneglycol and polyvinylalcohol, and the like may be mentioned.

According to another embodiment of the present disclosure, the composition of super absorbent polymer may further comprise one or more additives selected from the group consisting of metal iodide salt, organic acid and polyphenol, and by the additives, deodorization capacity and odor masking capacity may be further improved.

The metal iodide salt may be, for example, in the form wherein one or more selected from the group consisting of CuI, NaI and KI, and I₂ are dissolved in water together. The metal iodide salt may oxidize malodorous substance to confer deodorization property.

The organic acid may be, for example, one or more selected from the group consisting of citric acid, glutamic acid, ascorbic acid, benzoic acid and erythorbic acid.

The polyphenol may be, for example, one or more selected from the group consisting of tannin, anthocyanin, flavonol, isoflavon, catechin, polyquercetin, and caffeic acid.

The additives may be each independently included in the content of, preferably, 0.025 parts by weight or more, 0.05 parts by weight or more, 0.1 parts by weight or more, 2.5 parts by weight r less, 2.0 parts by weight or less, 1.5 parts by weight or less, or 0.01 to 2.5 parts by weight, 0.01 to 2.0 parts by weight, 0.025 to 2.5 parts by weight, 0.05 to 2.5 parts by weight, 0.05 to 2.0 parts by weight, 0.1 to 2.5 parts by weight, or 0.1 to 2.0 parts by weight, based on 100 parts by weight of the base resin.

If used in the above content range, they can realize synergistic effect with cysteine.

Meanwhile, in case the metal iodide salt is included in an excessive amount, unique properties of super absorbent polymer may be deteriorated, and it may be fed as a solution during the process to increase tailings, thus decreasing deodorization capacity to the contrary. The metal iodide salt is mixed with super absorbent polymer in the state of an aqueous solution with water as a solvent, and exists in the super absorbent polymer while being mixed and physically contained in the super absorbent polymer. The metal iodide salt may be a solution with a solid content concentration of 1% or less.

Meanwhile, the additives may be each independently included inside the surface crosslink layer or on the surface of the surface crosslink layer. It means that each additive is added during polymerization of base resin (i.e., before the surface crosslinking step), in the surface crosslinking step, or after the surface crosslinking step, which will be explained in detail in the preparation method of a composition of super absorbent polymer as described later.

### (Preparation Method of Composition of Super Absorbent Polymer)

According to one embodiment of the present disclosure, there is provided a method for preparing a composition of super absorbent polymer.

The method for preparing a composition of a superabsorbent polymer comprises steps of:
subjecting acrylic acid-based monomers having at least partially neutralized acid groups to crosslinking polymerization, in the presence of an internal crosslinking agent and a polymerization initiator, to form hydrogel polymer (step 1); preparing base resin comprising crosslinked polymer obtained by drying and grinding the hydrogel polymer (step 2); mixing a surface crosslinking composition with the base resin to prepare a mixture (step 3); and heat treating the mixture to prepare super absorbent polymer in which a surface crosslink layer is formed on the surface of the base resin (step 4), wherein an amino acetate-based chelating agent and cysteine are each independently mixed in at least one step of the steps 2 to 4 and steps before and after these steps.

The preparation method of a composition of super absorbent polymer can effectively control odor generated by various causes, without deteriorating basic properties of super absorbent polymer including absorption force and water holding capacity, by using an amino acetate-based chelating agent and cysteine in combination. To the amino acetate-based chelating agent and cysteine, the foregoing may be applied identically.

Meanwhile, the expression ‘mixed in step A’ means to be additionally mixed with a target mixture while the step A is conducted, and it may mean to be divided more than once and mixed at the content ratio aimed in corresponding step. Meanwhile, the expression ‘mixed before and after step A’ means to be additionally mixed before the step A is conducted or after the step A is finished.

More specifically, the amino acetate-based chelating agent and cysteine are each independently mixed in at least one step of the steps 2 to 4 and steps before and after these steps, which means that they are mixed during the surface crosslinking step, before the surface crosslinking step, or after the surface crosslinking step is finished, and they may be mixed in one or more steps. The two kinds of additives are included inside the surface crosslink layer or on the surface of the surface crosslink layer, and thereby, react with odor-generating substance and have excellent deodorization capacity, and inhibit bacteria growth to effectively inhibit additional odor generation. Meanwhile, in case the two kinds of additives are mixed in the polymerization step, it may be difficult to realize an aimed effect.

More preferably, the amino acetate-based chelating agent may be mixed in the step 2, or mixed after the step 4. More specifically, in case the chelating agent is included in the step of preparing base resin from polymerized hydrogel polymer (step 2), it means that it is be mixed with base resin and is added and mixed in the drying and grinding step. In this case, the chelating agent may be included inside the surface cross link layer formed in the surface crosslinking step. Further, in case the chelating agent is mixed after the surface crosslinking step (step 4), it means that it is mixed with super absorbent polymer having a surface crosslink layer. In this case, the chelating agent may be included on the surface of the surface crosslink layer formed in the surface crosslinking step.

More preferably, the cysteine may be mixed after the step 4. Specifically, in case the cysteine is mixed after the surface crosslinking step (step 4), it means that it is mixed with super absorbent polymer having a surface crosslink layer. In this case, the chelating agent may be included on the surface of the surface crosslink layer formed in the surface crosslinking step. In case the cysteine is included after the surface crosslinking step (step 4), it exists on the surface of super absorbent polymer, and thus, the contact area with odor may increase, which may be favorable in terms of improvement in deodorization capacity.

According to one embodiment of the present disclosure, in the preparation method of super absorbent polymer, one or more additives selected from the group consisting of metal iodide salt, organic acid and polyphenol may be further included, and to the additives, the foregoing may be applied identically.

The additives may be mixed in at least one step of the steps 2 to 4 and steps before and after these steps, which means that they are mixed during the surface crosslinking step, before the surface crosslinking step, or after the surface crosslinking step is finished, and they may be mixed in one or more steps. Thereby, the additives are included inside the surface crosslink layer or on the surface of the surface crosslink layer, and can more effectively inhibit odor generation.

More preferably, in case the additives are included after the surface crosslinking step (step 4), it means that they are mixed with super absorbent polymer having a surface crosslink layer. In this case, the additives exist on the surface of the surface crosslink layer formed in the surface crosslinking step, and thus, the contact area with odor increases, which may be favorable in terms of improvement in deodorization capacity.

Hereinafter, the disclosure will be explained in detail according to steps.

### (Step 1)

The step 1 is a step of preparing a hydrogel polymer, and specifically, a step wherein a monomer composition comprising acrylic acid-based monomers having at least partially neutralized acid groups is subjected to crosslinking polymerization to form a hydrogel polymer.

The acrylic acid-based monomers may be any monomers commonly used in the preparation of a superabsorbent polymer. Specifically, to the acrylic acid-based monomers, the foregoing may be applied identically.

Further, in the monomer composition, a polymerization initiator commonly used for the preparation of a super absorbent polymer may be included.

As the polymerization initiator, a thermal polymerization initiator or a photopolymerization initiator may be used according to polymerization method. However, even when photopolymerization is conducted, since a certain amount of heat is generated by UV irradiation, etc., and heat is generated to some degree according to the progression of an exothermic polymerization reaction, a thermal polymerization initiator may be additionally included.

As the photopolymerization initiator, one or more selected from the group consisting of benzoin ether, dialkyl acetophenone, hydroxyl alkylketone, phenyl glyoxylate, benzyl dimethyl Ketal, acyl phosphine, and α-aminoketone may be used. Among them, as specific examples of acyl phosphine, commercially available lucirin TPO, i.e., 2,4,6-trimethyl-benzoyl-trimethyl phosphine oxide may be used. More various photopolymerization initiators are described in Reinhold Schwalm, “UV Coatings: Basics, Recent Developments and New Application (Elsevier 2007)”, page 115, which may be referred to.

And, as the thermal polymerization initiator, one or more selected from the group consisting of a persulfate initiator, an azo initiator, hydrogen peroxide, and ascorbic acid may be used. Specific examples of the persulfate initiator may include sodium persulfate (Na₂S₂O₈), potassium persulfate (K₂S₂O₈), ammonium persulfate ((NH₄)₂S₂O₈), etc., and, specific examples of the azo initiator may include 2,2-azobis(2-amidinopropane) dihydrochloride, 2,2-azobis-(N,N-dimethylene) isobutyramidinedihydrochloride, 2-(carbamoylazo) isobutyronitril, 2,2-azobis[2-(2-imidazolin-2-yl)propane]dihydrochloride, 4,4-azobis-(4-cyanovalericacid), etc. More various thermal initiators are described in “Principle of Polymerization (Wiley, 1981)”, Odian, page 203, which may be referred to.

Such a polymerization initiator may be added at the concentration of 0.001 to 1 wt %, or 0.005 to 0.1 wt %, based on the monomer composition. If the concentration of the polymerization initiator is too low, polymerization speed may become slow, and remaining monomers may be extracted in a large quantity in the final product. And, if the concentration of the polymerization initiator is too high, polymer chains making up the network of a superabsorbent polymer may become short, and thus, extractable contents may increase, and absorbency under pressure of a superabsorbent polymer may be lowered, thereby deteriorating the properties of polymer.

Meanwhile, the polymerization of the monomer composition is conducted in the presence of an internal crosslinking agent to improve the properties of a polymer obtained by the polymerization of the acrylic acid-based monomers. To the internal crosslinking agent, the foregoing may be applied identically.

Further, the crosslinking polymerization of the monomer composition may be conducted in the presence of a blowing agent, according to the necessity and degree of improvement in absorption speed. Such a blowing agent may be decomposed during the crosslinking polymerization reaction and generate gas, thus forming pores in the hydrogel polymer. As the result, when such a blowing agent is additionally used, more developed porous structure is formed in a super absorbent polymer, and thus, absorption speed of super absorbent polymer may be further improved.

As non-limiting examples, the blowing agent may comprise one or more compounds selected from the group consisting of sodium bicarbonate, sodium carbonate, potassium bicarbonate, potassium carbonate, calcium bicarbonate, calcium carbonate, magnesium bicarbonate, magnesium carbonate, azodicarbonamide (ADCA), dinitroso pentamethylene tetramine (DPT), p,p′-oxybis(benzenesulfonyl hydrazide) (OBSH), p-toluenesulfonyl hydrazide (TSH), sucrose stearate, sucrose palmitate, and sucrose laurate.

The blowing agent may be included in the monomer composition in the content of 1000 to 4000 ppmw, and more specifically, in the content of 1000 ppm or more, or 1100 ppmw or more, or 1200 ppmw or more; and 4000 ppmw or less, or 3500 ppmw or less, or 3000 ppmw or less.

Besides, the monomer composition may further comprise a thickener, a plasticizer, a preservation stabilizer, an antioxidant, and the like, as necessary.

Further, the monomer composition may be prepared in the form of a solution in which the above-explained raw materials including acrylic acid-based monomers, polymerization initiator, internal crosslinking agent, blowing agent, and the like are dissolved in a solvent.

Wherein, the solvent that can be used is not limited so long as it can dissolve the above explained raw materials. For example, as the solvent, water, ethanol, ethyleneglycol, diethyleneglycol, triethyleneglycol, 1,4-butanediol, propyleneglycol, ethyleneglycol monobutyl ether, propyleneglycol monomethyl ether, propyleneglycol monomethyl ether acetate, methylethylketone, acetone, methylamylketone, cyclohexanone, cyclopentanone, diethyleneglycol monomethyl ether, diethyleneglycol ethyl ether, toluene, xylene, butyrolactone, carbitol, methylcellosolve acetate, N,N-dimethylacetamide, or a mixture thereof may be used.

The formation of hydrogel polymer through the polymerization of the monomer composition may be conducted by a common polymerization method, and the process is not specifically limited.

As non-limiting examples, the polymerization method is largely classified into thermal polymerization and photopolymerization according to the kind of energy source, and thermal polymerization may be progressed in a reactor equipped with a stirring axis such as a kneader, and photopolymerization may be progressed in a reactor equipped with a movable conveyer belt.

For example, hydrogel polymer may be obtained by adding the above-described monomer composition into a reactor such as a kneader equipped with a stirring axis, and supplying hot air or heating the reactor to progress thermal polymerization. Wherein, the hydrogel polymer discharged to the outlet of the reactor may be in the size of a few centimeters to a few millimeters according to the shape of the stirring axis equipped in the reactor. Specifically, the size of obtained hydrogel polymer may vary according to the concentration of the added monomer composition and the addition speed, etc., and commonly, a hydrogel polymer with particle diameter of 2 to 50 mm may be obtained.

For another example, in case photopolymerization of the monomer composition is progressed in a reactor equipped with a movable conveyer belt as explained above, hydrogel polymer in the form of a sheet may be obtained. Wherein, the thickness of the sheet may vary according to the concentration of the added monomer composition and the addition speed, but commonly, it is preferable that the thickness of the sheet is controlled to 0.5 to 10 cm, to enable uniform polymerization of the entire sheet, and simultaneously, secure production speed, and the like.

The hydrogel polymer thus obtained may have a moisture content of 40 to 80 wt %. Wherein, the “moisture content” is the content of moisture occupied based on the total weight of hydrogel polymer, and it means a value obtained by subtracting the weight of polymer of a dry state from the weight of hydrogel polymer. Specifically, it is defined as a value calculated by measuring the weight loss according to moisture evaporation in the polymer while raising the temperature of polymer through infrared heating to dry. Wherein, the temperature is raised from room temperature to about 180° C. and then maintained at 180° C., and the total drying time may be set to 20 minutes including a temperature raising step of 5 minutes.

### (Step 2)

The step 2 of the present disclosure is a step of drying and grinding the hydrogel polymer prepared in step 1, to form base resin.

Specifically, to increase drying efficiency of the hydrogel polymer and affect the morphology of the super absorbent polymer, thus affecting various properties of super absorbent polymer including absorption speed, particularly to improve absorption speed of super absorbent polymer, in the present disclosure, a step of coarse grinding may be further included before drying the hydrogel polymer. Hereinafter, to distinguish it from grinding after drying, the grinding before drying is referred to as ‘coarse grinding’ herein for convenience.

The grinder used for the grinding is not limited, but specifically, it may include any one selected from the group consisting of vertical pulverizer, turbo cutter, turbo grinder, rotary cutter mill, cutter mill, disc mill, shred crusher, crusher, chopper and disc cutter, but is not limited thereto.

Wherein, the coarse grinding step may be conducted such that the particle diameter of hydrogel polymer may become about 2 mm to about 10 mm. Grinding to particle diameter less than 2 mm would not be technically easy due to high moisture content of hydrogel polymer, and it may cause aggregation between ground particles. Meanwhile, if grinding to particle diameter greater than 10 mm, the effect for increasing the efficiency of the subsequent drying step may be insignificant.

The drying may be conducted at a temperature of 120 to 250° C., 140 to 200° C., or 150 to 190° C. Wherein, the drying temperature may be defined as the temperature of a heat transfer medium supplied for drying or the temperature inside a drying reactor containing a heat transfer medium and polymer in the drying process. If the drying temperature is too low and drying time is lengthened, process efficiency may be deteriorated, and thus, to prevent the same, the drying temperature is preferably 120° C. or more. And, if the drying temperature is higher than needed, the surface of hydrogel polymer may be excessively dried, and thus, generation of fine powders may increase in the subsequent grinding step, and the properties of the final polymer may be deteriorated, and thus, to prevent the same, the drying temperature is preferably 250° C. or less.

Wherein, the drying time in the drying step is not specifically limited, but considering process efficiency and properties of the polymer, and the like, it may be controlled to 20 minutes to 90 minutes under the above-described drying temperature.

The drying may be conducted using a common medium, and for example, the drying may be conducted by hot air supply, infrared irradiation, microwave irradiation or UV irradiation to the ground hydrogel polymer, and the like.

Further, preferably, drying is conducted such that dried polymer may have a moisture content of 0.1 to 10 wt %. Namely, in case the moisture content of dried polymer is less than 0.1 wt %, due to excessive drying, production cost may increase and crosslinked polymer may be degraded. And, if the moisture content of dried polymer is greater than 10 wt %, defects may be generated in the subsequent process.

Subsequently, the dried hydrogel polymer may be ground. It is a step for optimizing the surface area of base resin powder and super absorbent polymer. The grinding may be conducted such that the particle diameter of ground polymer may become 150 to 850 μm.

Wherein, as a grinder, common grinders such as a pin mill, a hammer mill, a screw mill, a roll mill, a disc mill, a jog mill, and the like may be used.

Further, to manage the properties of the finally productized super absorbent polymer, a step of selectively classifying particles having particle diameter range of 150 to 850 μm in the polymer particles obtained through the grinding step, is conducted.

Passing through the classification step, base resin may be obtained. Such base resin may have particle diameter of 150 to 850 μm, and may comprise fine powders with particle diameter less than 150 μm in the content of 2 wt % or less, or 1 wt % or less.

Meanwhile, as explained above, the amino acetate-based chelating agent may be included in the step of preparing base resin from polymerized hydrogel polymer (step 2), and in this case, aggregation of base resin may be prevented, and thus, property deterioration may be inhibited and the chelating agent may be distributed more uniformly. Meanwhile, the mixing method of the amino acetate-based chelating agent is not specifically limited, and it may be mixed by simply mixing, or spraying in the state of an aqueous solution.

### (Step 3 and Step 4)

The step 3 of the present disclosure is a step of mixing the base resin prepared in the step 2 with a surface crosslinking composition, and the step 4 is a step of heat treating the mixture to prepare a super absorbent polymer in which a surface crosslink layer is formed on the surface of the base resin.

The surface crosslinking composition used in step 3 comprises a surface crosslinking agent, and the surface crosslinking agent is not specifically limited so long as it is commonly used for surface crosslinking of super absorbent polymer and is a compound capable of reacting with the functional groups of the polymer.

Preferably, to improve the properties of produced super absorbent polymer, as the surface crosslinking agent, one or more selected from the group consisting of polyhydric alcohol compounds; epoxy compounds; polyamine compounds; haloepoxy compounds; condensation products of haloepoxy compounds; oxazoline compounds; mono-, di- or polyoxazolidinone compounds; cyclic urea compounds; multivalent metal salts; and alkylene carbonate compounds may be used.

Specifically, as the examples of the polyhydric alcohol compound, one or more selected from the group consisting of mono-, di-, tri-tetra- or polyethylene glycol, monopropylene glycol, 1,3-propanediol, dipropylene glycol, 2,3,4-trimethyl-1,3-pentanediol, polypropylene glycol, glycerol, polyglycerol, 2-butene-1,4-diol, 1,4-butanediol, 1,3-butanediol, 1,5-pentanediol, 1,6-hexandiol, and 1,2-cyclohexanedimethanol may be used.

Further, as the epoxy compound, ethylene glycol diglycidyl ether and glycidol, and the like may be used, and as the polyamine compound, one or more selected from the group consisting of ethylenediamine, diethylenetriamine, triethylenetriamine, tetraethylenepentamine, pentaethylenehexamine, polyethyleneamine, and polyamidepolyamine may be used.

Further, as the haloepoxy compound, epichlorohydrin, epibromohydrin, and α-methylepichlorohydrin may be used, and as the mono-, di- or polyoxazolidinone compound, for example, 2-oxazolidinone, and the like may be used.

Further, as the alkylene carbonate compound, ethylene carbonate, and the like may be used. These compounds may be used alone or in combinations. Meanwhile, to increase the efficiency of the surface crosslinking process, among these surface crosslinking agents, one or more kinds of C2-10 polyhydric alcohol compounds may be included.

The content of the surface crosslinking agent added may be appropriately selected according to the kind of the surface crosslinking agent added and reaction conditions, but commonly, it may be used in the amount of about 0.001 to about 5 parts by weight, preferably about 0.01 to about 3 parts by weight, more preferably about 0.05 to about 2 parts by weight, based on 100 parts by weight of the polymer.

If the content of the surface crosslinking agent is too small, a surface crosslinking reaction may hardly occur, and if it is greater than 5 parts by weight, based on 100 parts by weight of the polymer, due to the progression of excessive surface crosslinking reaction, absorption capacity and properties may be deteriorated.

Meanwhile, the step of forming a surface crosslink layer may be conducted while the surface crosslinking composition further comprises inorganic materials. As such inorganic material, one or more selected from the group consisting of silica, clay, alumina, silica-alumina composite material, titania, zinc oxide and aluminum sulfate may be used. The inorganic material may be used in the form of powder or liquid, and particularly, alumina powder, silica-alumina powder, titania powder, or a nano silica solution. And, the inorganic material may be used in the content of about 0.001 to about 1 part by weight, based on 100 parts by weight of the base resin.

Further, the surface crosslinking composition may further comprise a thickener. If the surface of base resin powder is additionally crosslinked in the presence of a thickener, property deterioration may be minimized even after grinding. Specifically, as the thickener, one or more selected from polysaccharide and hydroxy-containing polymer may be used. As the polysaccharide, gum-based thickener and cellulose-based thickener, and the like may be used. As specific examples of the gum-based thickener, xanthan gum, arabic gum, karaya gum, tragacanth gum, ghatti gum, guar gum, locust bean gum and psyllium seed gum, and the like may be mentioned, and as specific examples of the cellulose-based thickener, hyrdoxypropylmethylcellulose, carboxymethylcellulose, methylcellulose, hydroxymethylcellulose, hydroxyethylcellulose, hydroxypropylcellulose, hydroxyethylmethylcellulose, hydroxymethylpropylcellulose, hydroxyethylhydroxypropylcellulose, ethylhydroxyethylcellulose and methylhydroxypropylcellulose, and the like may be mentioned. Meanwhile, as specific examples of the hydroxy-containing polymer, polyethyleneglycol and polyvinylalcohol, and the like may be mentioned.

Meanwhile, a method for mixing the base resin with the surface crosslinking composition may be appropriately selected without specific limitations, so long as it can uniformly mix them.

For example, a method of putting the surface crosslinking composition and the base resin in a reactor and mixing them, a method of spraying the surface crosslinking composition to the surface of the base resin, a method of continuously feeding the base resin and the surface crosslinking composition to a continuously operated mixer and mixing, and the like, may be used.

Wherein, the surface crosslinking composition may be a solution, and in case the solid content in the solution is 1 wt % or more, 3 wt % or more, 5 wt % or more, 10 wt % or more, and 50 wt % or less, 30 wt % or less, or 20 wt % or less, it may be appropriate to uniformly disperse in the base resin, and simultaneously agglomeration of the base resin may be prevented.

Next, the step 4 is a step of heat treating the mixture to prepare super absorbent polymer in which a surface crosslink layer is formed on the surface of the base resin, and specifically, the base resin and a surface crosslinking composition are heat treated to form an interpenetrating polymer network on the surface of the crosslinked polymer included in the base resin, thereby further improving the properties of super absorbent polymer. Through such surface modification, on the surface of the ground base resin particles, a surface crosslink layer is formed.

The formation of the surface crosslink layer may be conducted by a common method of increasing the crosslinking density of polymer particle surface, and for example, it may be conducted by mixing the ground polymer with a surface crosslinking solution comprising a surface crosslinking agent, and heat treating the mixture to subject it to a crosslink reaction.

The step 4 may be conducted at a temperature of about 80° C. to about 250° C. More specifically, the surface crosslinking process may be conducted at a temperature of about 100° C. to about 220° C., or about 110° C. to about 200° C., or about 120° C. to about 190° C., for about 10 minutes to about 2 hours, or about 20 minutes to about 60 minutes. If the crosslinking reaction temperature is less than 160° C. or the reaction time is too short, a surface crosslinking reaction may not sufficiently occur, and thus, penetration degree may be lowered, and if the crosslinking reaction temperature is greater than 200° C. or the reaction time is too long, water holding capacity may be lowered.

A temperature rise means for the surface crosslinking reaction is not specifically limited. A heat transfer medium may be supplied, or a heat source may be directly supplied for heating. Wherein, as the kind of heat transfer media that can be used, temperature-increased fluids such as steam, hot air, hot oil, and the like may be used, but the present disclosure is not limited thereto, and the temperature of heat transfer medium supplied may be appropriately selected considering the means of heat transfer medium, temperature rise means, and target temperature. Meanwhile, as the heat source directly supplied, electric heating, and gas heating may be mentioned, but the present disclosure is not limited thereto.

Meanwhile, as explained above, the amino acetate-based chelating agent may be mixed after the step of heat treating the mixture to prepare super absorbent polymer in which a crosslink layer is formed on the surface of the base resin (step 4), which is favorable in that a possibility of generating tailings when treated with cysteine, and the like, after surface crosslinking, may be further lowered. Meanwhile, the mixing method of the amino acetate-based chelating agent is not specifically limited, and it may be mixed by simply mixing or spraying in the state of an aqueous solution.

Further, as explained above, the cysteine may be mixed after the step of heat treating the mixture to prepare super absorbent polymer in which a crosslink layer is formed on the surface of the base resin (step 4), which is favorable in that it exists on the surface of super absorbent polymer to increase contact area with odor, thereby improving deodorization capacity. Meanwhile, the mixing method of the cysteine is not specifically limited, and it may be mixed by spraying in the state of an aqueous solution or dry mixing.

Further, one or more additives selected from the group consisting of metal iodide salt, organic acid and polyphenol may be mixed after the step of heat treating the mixture to prepare super absorbent polymer in which a crosslink layer is formed on the surface of the base resin (step 4), which is favorable in that they exist on the surface of super absorbent polymer to increase contact area with odor, thereby improving deodorization capacity. Meanwhile, the mixing method of the additives is not specifically limited, and it may be mixed by spraying in the state of an aqueous solution or dry mixing.

According to one embodiment of the present disclosure, after the step of preparing super absorbent polymer in which a crosslink layer is formed on the surface of the base resin (step 4), a step of mixing amino acetate-based chelating agent, cysteine, metal iodide salt, organic acid and polyphenol in the form of aqueous solutions (step of addition after hydration) may be included, and in this case, a drying step may be additionally conducted. To the drying process, the foregoing may be applied identically.

Hereinafter, preferable examples are presented to assist in understanding of the disclosure. However, the following examples are presented only as the illustrations of the present disclosure and are not intended to limit the present disclosure.

### Examples and Comparative Examples: Preparation of Super Absorbent Polymer

### Example 1

(Step 1) 100 g of acrylic acid, 0.37 g of N,N′-methylenebisacrylamide as a crosslinking agent, 0.15 g of sodium persulfate (SPS) as a thermal initiator, 0.008 g of benzoin ether as UV initiator, 40 g of caustic soda (NaOH), and 127 g of water were mixed, to prepare an aqueous monomer composition with a monomer concentration of 45.8 wt %. The aqueous monomer composition was introduced into a feed section of a polymerization reactor equipped with a continuously moving conveyor belt, and then, while maintaining a polymerization atmosphere temperature of 80° C., UV was irradiated with a UV irradiation device (dose: 10 mW/cm²), and UV polymerization was progressed for 2 minutes, thus preparing a hydrogel polymer.

(Step 2) The hydrogel polymer was transferred to a meat chopper and cut to 2 mm to 10 mm. Wherein, the moisture content of cut hydrogel polymer was 47 wt %. Subsequently, the hydrogel polymer was dried in a hot air dryer of 170° C. for 30 minutes, and the dried hydrogel polymer was ground with a pin mill. Subsequently, polymers having particle sizes (average particle size) of 150 μm to 850 μm were classified with a sieve to prepare a base resin.

(Step 3) To 100 parts by weight of the above-prepared base resin, a surface crosslinking solution (2.5 parts by weight of water, 0.1 parts by weight of ethyleneglycol diglycidyl ether (EX-810), 0.1 parts by weight of aluminum sulfate 18 hydrate (AI-S), and 0.1 parts by weight of silica (Aerosil A200)) was uniformly mixed.

(Step 4) Next, a surface crosslinking reaction was progressed at 140° C. for 30 minutes. After completing the surface treatment, by classification with a sieve, super absorbent polymer with an average particle diameter of 150 to 850 μm was obtained. In the super absorbent polymer thus obtained, the content of polymer having average particle size less than 150 μm was less than 2%.

In the step 2, as the amino acetate-based chelating agent, EDTA was mixed by dissolving in an aqueous solution to the concentration of 0.5 parts by weight (based on 100 parts by weight of base resin), and spraying to the base resin.

Further, after the surface crosslinking of the step 4, L-cysteine was mixed by dissolving in an aqueous solution to the concentration of 0.2 parts by weight (based on 100 parts by weight of base resin), and spraying to the super absorbent polymer. Thereafter, a drying step was conducted to prepare a composition of super absorbent polymer.

### Examples 2 to 17 and Comparative Examples 1 to 3

Super absorbent polymers were prepared by the same method as Example 1, except that components, contents and addition times of the additives in Example 1 were changed as described in Table 1.

Meanwhile, in Examples 9 to 11, additional additives were sprayed in the form of aqueous solutions (metal iodide salt solution (1% concentration, CuI), tannin solution, citric acid solution) to the surface-crosslinked super absorbent polymer, and additional drying step was conducted.

TABLE 1

| | Chelating agent | | | Amino acid | | | Other additives | | |
|---|---|---|---|---|---|---|---|---|---|
| | Kind | Content | Addition time | Kind | Content | Addition time | Kind | Content | Addition time |
| Example 1 | EDTA | 0.5 | Step 2 | L-cysteine | 0.2 | After step 4 | — | — | — |
| Example 2 | EDTA | 0.1 | Step 2 | L-cysteine | 0.2 | After step 4 | — | — | — |
| Example 3 | GLDA | 0.5 | Step 2 | L-cysteine | 0.2 | After step 4 | — | — | — |
| Example 4 | GLDA | 1.0 | Step 2 | L-cysteine | 0.2 | After step 4 | — | — | — |
| Example 5 | EDTA | 0.5 | Step 2 | L-cysteine | 0.1 | After step 4 | — | — | — |
| Example 6 | EDTA | 0.5 | Step 2 | L-cysteine | 0.5 | After step 4 | — | — | — |
| Example 7 | EDTA | 0.5 | Step 2 | L-cysteine | 1.5 | After step 4 | — | — | — |
| Example 8 | EDTA | 0.5 | After step 4 | L-cysteine | 0.2 | After step 4 | — | — | — |
| Example 9 | EDTA | 0.5 | Step 2 | L-cysteine | 0.2 | After step 4 | Metal iodide salt | 1.0 | After step 4 |
| Example 10 | EDTA | 0.5 | Step 2 | L-cysteine | 0.2 | After step 4 | Tannin | 1.0 | After step 4 |
| Example 11 | EDTA | 0.5 | Step 2 | L-cysteine | 0.2 | After step 4 | Citric acid | 1.0 | After step 4 |
| Example 12 | EDTA | 0.25 | Step 2 | L-cysteine | 0.2 | After step 4 | — | — | — |
| Example 13 | GLDA | 0.25 | Step 2 | L-cysteine | 0.2 | After step 4 | — | — | — |
| Example 14 | EDTA | 0.25 | Step 2 | L-cysteine | 0.05 | After step 4 | — | — | — |
| Example 15 | MGDA | 0.5 | Step 2 | L-cysteine | 0.2 | After step 4 | — | — | — |
| Example 16 | HEDTA | 0.5 | Step 2 | L-cysteine | 0.2 | After step 4 | — | — | — |
| Example 17 | EDG | 0.5 | Step 2 | L-cysteine | 0.2 | After step 4 | — | — | — |
| Example 18 | DTPA | 0.5 | Step 2 | L-cysteine | 0.2 | After step 4 | — | — | — |
| Comparative | EDTA | — | Step 2 | — | 0.2 | After step 4 | — | — | — |
| Example 1 | | | | | | | | | |
| Comparative | EDTA | 0.1 | Step 2 | — | 0.2 | After step 4 | — | — | — |
| Example 2 | | | | | | | | | |
| Comparative | — | — | — | L-cysteine | 0.2 | After step 4 | — | — | — |
| Example 3 | | | | | | | | | |
| Comparative | Metal salt of | 0.1 | Step 2 | L-cysteine | 0.2 | After step 4 | — | — | — |
| Example 4 | recinoleic acid | | | | | | | | |
| Comparative | EDTA | 0.25 | Step 2 | Methionine | 0.2 | After step 4 | — | — | — |
| Example 5 | | | | | | | | | |

### Experimental Example

For the compositions of super absorbent polymers prepared in Examples and Comparative Examples, the properties were measured as follows.

### (1) Evaluation of Absorption Properties

### 1) Centrifuge Retention Capacity (CRC)

Centrifuge retention capacity of each super absorbent polymer composition of Examples and Comparative Examples by absorption rate under no load was measured according to European Disposables and Nonwovens Association, (EDANA) standard EDANA WSP 241.3.

Specifically, from each super absorbent polymer composition obtained through Examples and Comparative Examples, polymers classified with sieves of #30-50 were obtained. W₀ (g, about 0.2 g) of such polymer was uniformly put in an envelope made of non-woven fabric, and the envelope was sealed, and then, dipped in a 0.9 wt % saline solution. After 30 minutes, the envelope was drained at 250 G for 3 minutes using a centrifuge, and the weight W₂ (g) of the envelope was measured. And, the same operation was conducted using an empty envelope without polymer, and then, the weight W₁ (g) at that time was measured.

Using the weights thus obtained, CRC (g/g) was confirmed by the following Mathematical Formula 1.

$$CRC ( g / g ) = \{ [ {W}_{2} ( g ) - {W}_{1} ( g ) ] / {W}_{0} ( g ) \} - 1 \qquad [ Mathematical Formula 1 ]$$

### 2) AUP: Absorbency Under Pressure (AUP)

For each super absorbent polymer composition of Examples and Comparative Examples, 0.7 psi absorbency under pressure was measured according to EDANA method WSP 242.3.

First, when measuring absorbency under pressure, the classified polymer used to measure CRC was used.

Specifically, on the bottom of a plastic cylinder having an inner diameter of 25 mm, a 400 mesh wire netting made of stainless steel was installed. And, W₀ (g) of the super absorbent polymer was uniformly distributed on the screen under conditions of room temperature and 50% humidity, and a piston capable of uniformly applying a load of 0.7 psi was put thereon, wherein a piston having an outer diameter slightly smaller than 25 mm was used such that there was no gap with the inner wall of the cylinder and the up and down movement was not hindered. At this time, the weight W₃ (g) of the apparatus was measured.

Inside a petri dish having a diameter of 150 mm, a glass filter having a diameter of 90 mm and a thickness of 5 mm was laid, and a 0.9 wt % saline solution was poured to the same level as the upper side of the glass filter. And, one piece of a filter paper having a diameter of 90 mm was laid thereon. And then, the above measurement apparatus was put on the filter paper, and the solution was absorbed under pressure for 1 hour. After 1 hour, the measurement apparatus was lifted, and the weight W₄ (g) was measured. Using the obtained weights, absorbency under pressure (g/g) was calculated according to the following Mathematical Formula 2.

$$AUP ( g / g ) = [ {W}_{4} ( g ) - {W}_{3} ( g ) ] / {W}_{0} ( g ) \qquad [ Mathematical Formula 2 ]$$

### (2) Evaluation of Bacteria Inhibition Rate

Bacteria inhibition rate of each super absorbent polymer composition of Examples and Comparative Examples was evaluated as follows.

Specifically, commercial bacteria of E. coli (Escherichia Coli ATCC25922) were cultured in LB-Broth for 24 hours, and then, the cultured bacteria were diluted and injected in 2 g of each sample of the super absorbent polymer composition together with artificial urine, and it was cultured at 37° C. for 24 hours, and then, bacteria were extracted, and the number of bacteria was measured (mean value of 2 specimens).

Bacteria inhibition rate (%)=(1−((bacteria amount of sample)/bacteria amount of (Reference sample (sample without deodorization capacity)))×100 (%)

The experiment results of the bacteria inhibition rate were shown in Table 2. The numerical values of Table 2 indicate bacteria inhibition rate, and the higher the bacteria inhibition rate, the higher the numerical value.

### (3) Deodorization Capacity (Deodorization Rate)

Deodorization capacity was measured by an adsorption tube measurement method. Ketone (diacetyl), sulfur compound (dimethyl trisulfide) and aldehyde (3-methyl butanal) were selected as malodorous substances and deodorization capacity was tested.

Adsorption tube measurement method: Into a 500 mL glass bottle, 1 g of super absorbent polymer was put, and then, 25 mL of malodorous substance was introduced. And then, aging was progressed in a constant temperature chamber for 3 hours, followed by capturing for 20 minutes. Wherein, the temperature of the constant temperature chamber was 35° C., and N2 flow rate was 230 ml/min. The odor pushed out was then adsorbed to the connected adsorption tube, which was repeated twice per the same sample to capture. The result of capture was confirmed by GC analysis.

$$Deodorization rate ( \% ) = the amount of odor of Reference \\ sample ( sample without deodorization capacity ) measured by GC - the amount of odor of sample measured \\ by GC / the amount of odor of Reference sample \\ ( sample without deodorization capacity ) measured by GC \times 100 ( \% )$$

The experiment results of deodorization capacity were shown in Table 2. The numerical values of Table 2 indicate deodorization efficiencies compared to Comparative Example 1, and the higher the numerical value the higher the deodorization efficiency.

TABLE 2

| | | Deodorization capacity | | | | |
|---|---|---|---|---|---|---|
| | Bacteria | | | Sulfur | Absorption properties | |
| | inhibition | Aldehyde | Ketone | compound | CRC | 0.7 AUP |
| | rate (%) | (%) | (%) | (%) | (g/g) | (g/g) |
| Example 1 | 99 | 90 | 90 | 85 | 31.0 | 22.0 |
| Example 2 | 99 | 94 | 94 | 87 | 30.5 | 21.7 |
| Example 3 | 99 | 95 | 92 | 83 | 31.2 | 22.0 |
| Example 4 | 99 | 90 | 90 | 85 | 31.0 | 22.3 |
| Example 5 | 99 | 90 | 90 | 82 | 31.7 | 21.7 |
| Example 6 | 99 | 90 | 90 | 93 | 31.2 | 22.2 |
| Example 7 | 99 | 90 | 90 | 98 | 30.1 | 20.2 |
| Example 8 | 99 | 90 | 90 | 93 | 31.0 | 22.0 |
| Example 9 | 99 | 94 | 93 | 98 | 30.2 | 22.1 |
| Example 10 | 99 | 98 | 97 | 98 | 30.7 | 21.5 |
| Example 11 | 99 | 95 | 93 | 90 | 30.0 | 20.8 |
| Example 12 | 90 | 75 | 67 | 83 | 31.0 | 22.2 |
| Example 13 | 98 | 88 | 85 | 85 | 31.6 | 21.9 |
| Example 14 | 85 | 75 | 67 | 67 | 32.0 | 21.8 |
| Example 15 | 99 | 92 | 90 | 85 | 30.1 | 20.2 |
| Example 16 | 99 | 90 | 90 | 80 | 31.2 | 22.0 |
| Example 17 | 99 | 90 | 90 | 85 | 30.2 | 21.1 |
| Example 18 | 99 | 98 | 97 | 83 | 30.1 | 21.5 |
| Comparative | 0 | 0 | 0 | 0 | 32.3 | 22.5 |
| Example 1 | | | | | | |
| Comparative | 85 | 0 | 0 | 0 | 32.0 | 22.7 |
| Example 2 | | | | | | |
| Comparative | 0 | 90 | 85 | 82 | 31.0 | 21.5 |
| Example 3 | | | | | | |
| Comparative | 35 | 75 | 80 | 78 | 30.5 | 20.5 |
| Example 4 | | | | | | |
| Comparative | 98 | 97 | 63 | 45 | 30.0 | 21.0 |
| Example 5 | | | | | | |

From the results of Table 2, it can be confirmed that the compositions of super absorbent polymer according to Examples of the present disclosure, by using amino acetate-based chelating agent and cysteine in combination, exhibit excellent absorption properties, and simultaneously, have excellent deodorization capacity and odor masking capacity, and can effectively inhibit bacteria growth during wearing of a product.

It can be confirmed that in the case of Comparative Examples wherein only a part of the two additives was used, it was difficult to simultaneously realize bacteria inhibition rate and deodorization capacity. Particularly, in case a metal salt of ricinoleic acid was used instead of the amino acetate-based chelating agent in combination with cysteine, although deodorization capacity was similar to those of Examples, bacteria growth inhibition rate was remarkably low.

1. A composition of super absorbent polymer comprising:
a super absorbent polymer comprising
a base resin including a crosslinked polymer formed by crosslinking polymerization of acrylic acid-based monomers having at least partially neutralized acid groups and an internal crosslinking agent, and
a surface crosslink layer formed on a surface of the base resin, wherein
the crosslinked polymer is further crosslinked by a surface crosslinking agent;
an amino acetate-based chelating agent; and
cysteine,
wherein the amino acetate-based chelating agent and the cysteine are each independently included inside the surface crosslink layer or on a surface of the surface crosslink layer.

2. The composition of claim 1, wherein the amino acetate-based chelating agent includes one or more of ethylenediamine tetraacetic acid (EDTA), L-glutamic acid diacetate (GLDA), methyl glycine diacetic acid (MGDA), hydroxyethyl ethylenediamine triacetic acid (HEDTA), ethanol diglycinic acid (EDG), diethylenetriamine pentaacetic acid (DTPA), and or salts thereof.

3. The composition of claim 1, wherein the amino acetate-based chelating agent is included in an amount ranging from 0.01 to 3 parts by weight, based on 100 parts by weight of the base resin.

4. The composition of claim 1, wherein the cysteine is included in an amount ranging from 0.01 to 3 parts by weight, based on 100 parts by weight of the base resin.

5. The composition of claim 1, further comprising one or more additives of metal iodide salt, organic acid or polyphenol.

6. A method for preparing a composition for a super absorbent polymer, comprising:
subjecting acrylic acid-based monomers having at least partially neutralized acid groups to crosslinking polymerization, in the presence of an internal crosslinking agent and a polymerization initiator, to form a hydrogel polymer;
preparing a base resin comprising a crosslinked polymer obtained by drying and grinding the hydrogel polymer;
mixing a surface crosslinking composition with the base resin to prepare a mixture; and
heat treating the mixture to prepare the super absorbent polymer, wherein a surface crosslink layer is formed on a surface of the base resin,
wherein an amino acetate-based chelating agent and cysteine are each independently mixed in at least one steps of the preparing the base resin, the mixing the surface crosslinking composition with the base resin, or the heat treating the mixture or steps before or after these steps.

7. The method of claim 6, wherein the amino acetate-based chelating agent is mixed in the preparing of the base resin, or mixed after the heat treating the mixture.

8. The method of claim 6, wherein the cysteine is mixed after the heat treating the mixture.

9. The method of claim 6, further comprising mixing at least one additives of metal iodide salt, organic acid or polyphenol.

10. The method of claim 9, wherein the additives is each independently mixed in at least one step of the preparing the base resin, the mixing the surface crosslinking composition with the base resin, or the heat treating the mixture or steps before or after these steps.

11. The method of claim 9, wherein the additives is each independently mixed after the heat treating the mixture.
